(19) United States
(12) Patent Application Publication
(10) Pub. No.: US 2021/0222152 A1
(43) Pub. Date: Jul. 22, 2021

(54) METHOD TO EXTRACT CHROMATIN FROM FORMALIN FIXED, PARAFFIN EMBEDDED (FFPE) TISSUE

(71) Applicant: The University of North Carolina at Chapel Hill, Chapel Hill, NC (US)

(72) Inventors: Samantha Gail Pattenden, Mebane, NC (US); Paul Alexander Dayton, Carrboro, NC (US); Ian Jonathan Davis, Chapel Hill, NC (US); Austin Louis Quimby, Chapel Hill, NC (US)

(21) Appl. No.: 17/054,383

(22) PCT Filed: May 10, 2019
(86) PCT No.: PCT/US2019/031724

§ 371 Date: Nov. 10, 2020

Related U.S. Application Data
(60) Provisional application No. 62/669,715, filed on May 10, 2018.

(51) Int. Cl. C12N 15/10; C12Q 1/6806

(52) U.S. Cl. CPC C12N 15/1003; C12Q 1/6806

(57) ABSTRACT

Methods of extracting chromatin from tissue, such as formalin fixed, paraffin embedded (FFPE) tissue, are provided. The methods are rapid, simple, and preserve the chromatin signature. The methods can include, for example, removal of the tissue from the paraffin, enzymatic digestion of the extracellular matrix, and exposure to ultrasound energy, optionally in the presence of a cavitation enhancement agent, such as microbubbles, nanobubbles, and/or phase-change nanodroplets. The methods can also include a mechanical tissue dissociation step. The methods provide chromatin fragments that are free of enzyme bias from fragmentation by enzymes such as micrococcal nuclease (MNase) and that are of optimal size for further quantification and/or identification. Kits for extracting chromatin from tissue are also provided.

### PRIORITY CLAIM

This application claims the priority benefit of U.S. Provisional Patent Application Ser. No. 62/669,715, filed May 10, 2018, the disclosure of which is incorporated herein by reference in its entirety.

### GOVERNMENT INTEREST

This invention was made with government support under Grant Numbers CA206939 and TR001111 awarded by the National Institutes of Health. The government has certain rights in the invention.

### TECHNICAL FIELD

The subject matter described herein relates to methods and kits for extracting chromatin from tissue. More particularly, the subject matter described herein relates to methods and kits for extracting chromatin from formaldehyde fixed paraffin embedded (FFPE) tissue, including FFPE human tissue.

### BACKGROUND

Eukaryotic cell nuclei comprise chromatin, a complex of DNA and protein. The protein in chromatin can help to package the DNA in the nuclei and help control its functions. More particularly, chromatin includes repeating nucleosome units that contain two pairs of four types of histone proteins (H2A, H2B, H3 and H4) forming an octamer or eight-unit histone core, that is wrapped 1.65 turns by a 147-base length of DNA controlling access to the underlying sequence. The composition, modification and structure of chromatin plays an important role in gene expression and in several biological processes, including DNA replication and repair, apoptosis, development and pluripotency.

During interphase, chromatin accessibility is dynamic, to allow DNA and RNA polymerases to replicate and transcribe the DNA, respectively. DNA regions coding for genes that are actively being transcribed or that are part of a regulatory element such as an active enhancer are generally free of protein and can be referred to as “open” or “accessible,” as these regions are more accessible to transcription factors and other proteins. DNA regions that code for inactive genes or regulatory regions remain more tightly packed and associated with proteins. Thus, these “closed” or “inaccessible” regions contain nucleosomes and are, for example, generally resistant to DNaseI digestion.

A variety of chromatin accessibility assays have been used in the field to separate the genome by chemical or enzymatic means to isolate open or closed regions. For example, DNase assays and formaldehyde assisted isolation of regulatory elements (FAIRE) assays can isolate open regions, the assay for transposable accessible chromatin (ATAC) can isolate open regions as well as small regions between nucleosomes, micrococcal nuclease (MNase) assays can determine nucleosome position and indirectly show regions of open chromatin, and chromatin immunoprecipitation (ChIP) can determine protein occupancy at both open and closed regions of chromatin. The DNA isolated by these assays can then be quantified and/or identified by quantitative PCR (qPCR) or high throughput sequencing (HTS). These assays can be important tools to identify epigenetic changes related to differential gene expression, cell proliferation, and disease development. For instance, these assays can be used to distinguish diseased cells from healthy cells, and, therefore, could be helpful in diagnostic methods and in the development of new therapeutics.

Human clinical tissue samples are often preserved by fixation with formaldehyde and embedding in paraffin. Thus, there exists vast libraries of formaldehyde-fixed, paraffin embedded (FFPE) samples in hospitals and research laboratories that contain chromatin and that could yield large amounts of historical and/or population-wide data with regard to gene expression and disease development. However, while the afore-mentioned accessibility assays offer potential with regard to the analysis of these samples, current methods can be labor and/or time intensive, suffer from low signal to noise and/or require large numbers of cells. In particular, it can be difficult to extract chromatin from human FFPE tissue for use in these assays due to the larger amounts of materials present in the extracellular matrix of human tissue as compared to some other mammalian tissues. Current methods can also result in the extraction of chromatin-derived DNA fragments that are either too large or too small and/or degraded for reliable analysis.

Accordingly, there is an ongoing need for additional methods of extracting chromatin from tissue, particularly for additional methods that are useful for extracting chromatin from human tissues, particularly FFPE human tissue; that are easier and/or quicker to perform; and that can more consistently provide suitably sized, high quality DNA fragments that can be more readily quantified and/or identified.

### SUMMARY

In some embodiments, the presently disclosed subject matter provides a method of extracting chromatin from tissue, the method comprising: (a) receiving a sample comprising a biological tissue; (b) contacting the sample with an enzymatic solution, wherein the enzymatic solution comprises one or more enzymes that digest one or more extracellular matrix components; and (c) exposing the sample to ultrasound energy, thereby providing a processed sample comprising chromatin fragments that have been extracted from the biological tissue.

In some embodiments, the sample is from a formaldehyde fixed paraffin embedded (FFPE) tissue. In some embodiments, receiving the sample further comprises initially processing the sample, wherein the initial processing comprises performing a mechanical dissociation step, optionally wherein the mechanical dissociation step comprises bead beating or use of a tissue homogenizer or probe sonicator.

In some embodiments, the method provides a processed sample wherein chromatin fragments derived from accessible chromatin in the tissue sample are quantifiably distinguishable from chromatin fragments derived from inaccessible chromatin in the tissue sample or a processed sample wherein chromatin fragments derived from precipitation of a protein crosslinked to chromatin are distinguishable from chromatin fragments that do not contain the protein. In some embodiments, the amount of and/or the detectable signal generated from chromatin fragments derived from the accessible chromatin is at least 1.5 times or more than the amount of and/or detectable signal generated from chromatin fragments derived from the inaccessible chromatin, optionally wherein the chromatin fragments in the processed sample are assayed using quantitative PCR or high throughput sequencing.

In some embodiments, the presently disclosed subject matter provides a method of extracting chromatin from tissue, the method comprising: (a) receiving and initially processing a sample comprising a biological tissue, wherein the sample is from a formaldehyde fixed paraffin embedded (FFPE) tissue, and wherein initially processing the sample comprises contacting the FFPE tissue with an organic solvent; and (b) exposing the sample to ultrasound energy, thereby providing a processed sample comprising chromatin fragments that have been extracted from the biological tissue. In some embodiments, initially processing the sample further comprises performing a mechanical dissociation step, optionally wherein the mechanical dissociation step comprises bead beating or use of a tissue homogenizer or probe sonicator.

In some embodiments, the method provides a processed sample wherein chromatin fragments derived from accessible chromatin in the tissue sample are quantifiably distinguishable from chromatin fragments derived from inaccessible chromatin in the tissue sample or a processed sample wherein chromatin fragments derived from precipitation of a protein crosslinked to chromatin are distinguishable from chromatin fragments that do not contain the protein. In some embodiments, the amount of and/or the detectable signal generated from chromatin fragments derived from the accessible chromatin is at least 1.5 times or more than the amount of and/or detectable signal generated from chromatin fragments derived from the inaccessible chromatin, optionally wherein the chromatin fragments in the processed sample are assayed using quantitative PCR or high throughput sequencing.

In some embodiments, the presently disclosed subject matter provides a kit for extracting chromatin from a tissue, the kit comprising: (a) an extracellular matrix (ECM) digestion solution comprising one or more enzymes that digest an extracellular matrix component, optionally wherein the one or more enzymes comprise collagenase and/or hyaluronidase; and (b) a cavitation enhancement agent.

Accordingly, it is an object of the presently disclosed subject matter to provide methods of extracting chromatin from tissue and related kits.

An object of the presently disclosed subject matter having been stated hereinabove, and which is achieved in whole or in part by the presently disclosed subject matter, other objects will become evident as the description proceeds hereinbelow.

### BRIEF DESCRIPTION OF THE DRAWINGS

FIG. 1 is a schematic drawing showing a formaldehyde assisted isolation of regulatory elements (FAIRE) chromatin accessibility assay. Chromatin is first cross-linked with formaldehyde (top) and then sheared via sonication to form fragments of accessible/open regulatory DNA and fragments of inaccessible/closed DNA crosslinked to nucleosomes. The sheared mixture is digested with RNase enzyme to remove RNA. The RNase-digested sheared mixture can then be purified to isolate regulatory DNA elements (open/accessible chromatin), which can be analyzed via quantitative polymerase chain reaction (qPCR) or high throughput sequencing (HTS).

FIG. 2 is a schematic drawing showing an exemplary process for extracting chromatin from formalin fixed paraffin embedded (FFPE) tissue or frozen tissue as part of a formaldehyde assisted isolation of regulatory elements (FAIRE) chromatin accessibility assay optionally using nanodroplet assisted sonication.

FIG. 3 is a graph showing the percent yield of soluble chromatin extracted from rodent xenograft formalin fixed paraffin embedded (FFPE) tissue using sonication in the absence of nanodroplets (“Traditional”, filled diamonds) or in the presence of nanodroplets (“Nanodroplets”, unfilled diamonds) as a function of sonication time (in minutes (min)).

FIG. 4A is a series of graphs showing genome browser visualization of formaldehyde assisted isolation of regulatory elements (FAIRE) sequencing data (FAIRE-seq). The two graphs at the top are data from DNA isolated from tissue culture cells using nanodroplet assisted sonication; while the two graphs at the bottom are data from DNA isolated from formalin fixed paraffin embedded (FFPE) xenograft tissue derived from the same type of tissue culture cells using nanodroplet assisted sonication.

FIG. 4B is a graph showing the normalized read depth of formaldehyde assisted isolation of regulatory elements (FAIRE) sequencing signal ±3 kilo base pairs (bp) from the transcription start sites (TSS). Data is provided for signal from tissue culture cells processed using sonication without nanodroplet assistance (Traditional, thin solid line), tissue culture cells processed using nanodroplet assisted sonication (Nanodroplet, dotted and dashed line), and from two different formalin fixed paraffin embedded (FFPE) xenograft tissue blocks (FFPE Block 1 (dotted line) and FFPE Block 2 (thick solid line)).

FIG. 5 is a pair of graphs showing the effect of micrococcal nuclease (MNase) digestion on formaldehyde assisted isolation of regulatory elements (FAIRE) signal. The graph on the left shows FAIRE signal (as percent FAIRE/input signal) for DNA from a closed chromatin region (Negative Region) and two open chromatin regions (Promoter (Positive) Region; and Enhancer (Positive) Region) in samples that used MNase digestion, while the graph on the right shows signal for DNA from the same regions in samples processed without MNase but using nanodroplet assisted sonication (Nanodroplets).

FIG. 6 is a graph showing the effect of enzyme digestion prior to sonication with nanodroplets on formaldehyde assisted isolation of regulatory elements (FAIRE) signal (presented as percent FAIRE/input signal). Formalin fixed paraffin embedded (FFPE) tissue sections were treated with buffer (Nanodroplets only) or with an enzyme cocktail (Nanodroplets plus enzyme) prior to sonication in the presence of nanodroplets. All samples were subjected to FAIRE assay to isolate accessible chromatin followed by quantitative polymerase chain reaction (qPCR). Pre-processing of the tissue with enzyme digestion resulted in a higher FAIRE signal (POSITIVE 1, POSITIVE 2, POSITIVE 3, open chromatin regions) over background (NEGATIVE, closed chromatin region) ratio compared to no pre-processing step.

FIG. 7 is a graph showing the effect of different exemplary chromatin extraction methods on formaldehyde assisted isolation of regulatory elements (FAIRE) signal (provided as Percent FAIRE/Input signal). “Nanodroplets only” refers to a method wherein tissue is extracted using nanodroplet-assisted sonication; “Bead Beating+Nanodroplets” refers to a method that combines mechanical tissue disruption using bead beating and nanodroplet-assisted sonication; “Digestion 6 Hours+Nanodroplets” refers to a method wherein tissue is digested with an extracellular matrix digestion enzyme mixture for 6 hours prior to nanodroplet-assisted sonication; “Digestion 17 Hours+Nanodroplets” refers to a method wherein tissue is digested with an extracellular matrix digestion enzyme mixture for 17 hours prior to nanodroplet-assisted sonication; and “Digestion+Bead Beating+Nanodroplets” refers to a method that combines bead beating, extracellular matrix enzyme digestion and nanodroplet-assisted sonication. “Positive” is from an open chromatin region, while “Negative” is from a closed chromatin region.

FIG. 8A is a flow chart of an exemplary method for extracting chromatin from formalin fixed paraffin embedded (FFPE) tissue according to the presently disclosed subject matter.

FIG. 8B is a flow chart of another exemplary method for extracting chromatin from formalin fixed paraffin embedded (FFPE) tissue according to the presently disclosed subject matter.

FIG. 8C is a flow chart of another exemplary method for extracting chromatin from formalin fixed paraffin embedded (FFPE) tissue according to the presently disclosed subject matter.

FIG. 8D is a flow chart of another exemplary method for extracting chromatin from formalin fixed paraffin embedded (FFPE) tissue according to the presently disclosed subject matter.

### DETAILED DESCRIPTION

The presently disclosed subject matter will now be described more fully. The presently disclosed subject matter can, however, be embodied in different forms and should not be construed as limited to the embodiments set forth herein below and in the accompanying Figures and Examples. Rather, these embodiments are provided so that this disclosure will be thorough and complete, and will fully convey the scope of the embodiments to those skilled in the art.

All references listed herein, including but not limited to all patents, patent applications and publications thereof, and scientific journal articles, are incorporated herein by reference in their entireties to the extent that they supplement, explain, provide a background for, or teach methodology, techniques, and/or compositions employed herein.

### I. Definitions

While the following terms are believed to be well understood by one of ordinary skill in the art, the following definitions are set forth to facilitate explanation of the presently disclosed subject matter.

Unless defined otherwise, all technical and scientific terms used herein have the same meaning as commonly understood to one of ordinary skill in the art to which the presently disclosed subject matter belongs.

Following long-standing patent law convention, the terms “a”, “an”, and “the” refer to “one or more” when used in this application, including the claims.

The term “and/or” when used in describing two or more items or conditions, refers to situations where all named items or conditions are present or applicable, or to situations wherein only one (or less than all) of the items or conditions is present or applicable.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” can mean at least a second or more.

The term “comprising”, which is synonymous with “including,” “containing,” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

Unless otherwise indicated, all numbers expressing quantities of time, temperature, volume, diameter, percentage (%), and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about”, when referring to a value is meant to encompass variations of in one example ±20% or ±10%, in another example ±5%, in another example ±1%, and in still another example ±0.1% from the specified amount, as such variations are appropriate to perform the disclosed methods.

The term “bubble” as used herein refers to a bubble of gas that can, in some cases, be encased or surrounded by an enclosing substance. Bubbles that are from one micrometer to several tens or hundreds of micrometers in size are commonly referred to as “microbubbles”, while bubbles that are smaller than one micrometer in size are commonly referred to as “nanobubbles.”

The term “droplet” as used herein refers to an amount of liquid that is encased or surrounded by a different, enclosing substance. Droplets that are less than one micrometer in size are commonly referred to as “nanodroplets” and those that are in the one micrometer to tens or hundreds of micrometers in size are commonly referred to as “microdroplets.”

### II. Chromatin Extraction and Related Assays

In accordance with the subject matter disclosed herein, methods of extracting chromatin from tissue are provided. In some embodiments, the methods are used as part of a chromatin accessibility assay. Various different chromatin accessibility assays are known in the art, See Tsompana and Buck, Epigenetics & Chromatin, 2014, 7:33. For example, micrococcal nuclease (NMase) digestion assays, which use MNase to cleave chromatin between nucleosomes, provide DNA fragments associated with nucleosome positioning. DNase digestion assays and formaldehyde assisted interrogation of regulatory elements (FAIRE) assays both provide DNA fragments associated with open chromatin regions. The assay for transposable accessible chromatin (ATAC) also provides DNA fragments from open chromatin regions. In some embodiments, the presently disclosed subject matter relates to methods of extracting chromatin from tissue as part of a FAIRE-type assay. In addition, the presently disclosed subject matter can, in some embodiments, be used as part of other techniques involving chromatin extraction and fragmentation, such as, but not limited to chromatin immunoprecipitation (ChIP).

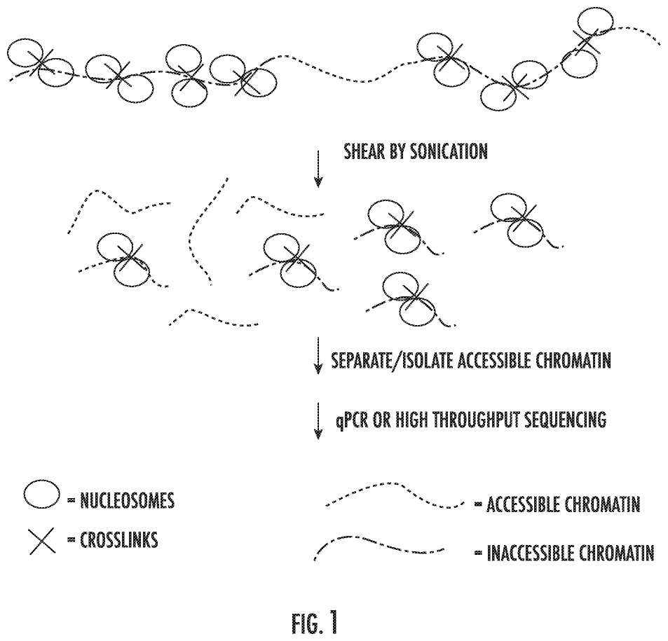

More particularly, unlike other chromatin accessibility assays that involve some form of enzymatic DNA cleavage, conventional FAIRE methods involve the crosslinking of nucleosomes in chromatin in vivo using formaldehyde followed by nucleic acid shearing via sonication. See FIG. 1. See also, Pattenden et al., PNAS, 113(11), 3018-3023 (2016); and Nagy et al, PNAS, 100(11), 6364-6369 (2003). Sonication of the crosslinked chromatin provides a mixture of two different types of fragments from the crosslinked chromatin: DNA fragments from open, regulatory DNA regions, and DNA fragments from closed, coding DNA regions that also include cross-linked nucleosomes. This mixture can be purified, e.g., via phenol-chloroform extraction of the open or free DNA fragments or via another suitable technique known in the art, such as by using a silica matrix column. The purified DNA fragments can then be detected via quantitative PCR (qPCR) or high throughput sequencing via a next generation sequencing method (FAIRE-seq). While FAIRE can overcome the sequence-specific cleavage biases associated with other chromatin accessibility assays, FAIRE can suffer from low signal-to-noise, making data interpretation difficult. In particular, oversonication can provide degraded DNA fragments and/or DNA fragments that are too short for optimal analysis. Oversonication can be especially problematic when dealing with human tissue samples and/or FFPE tissue samples.

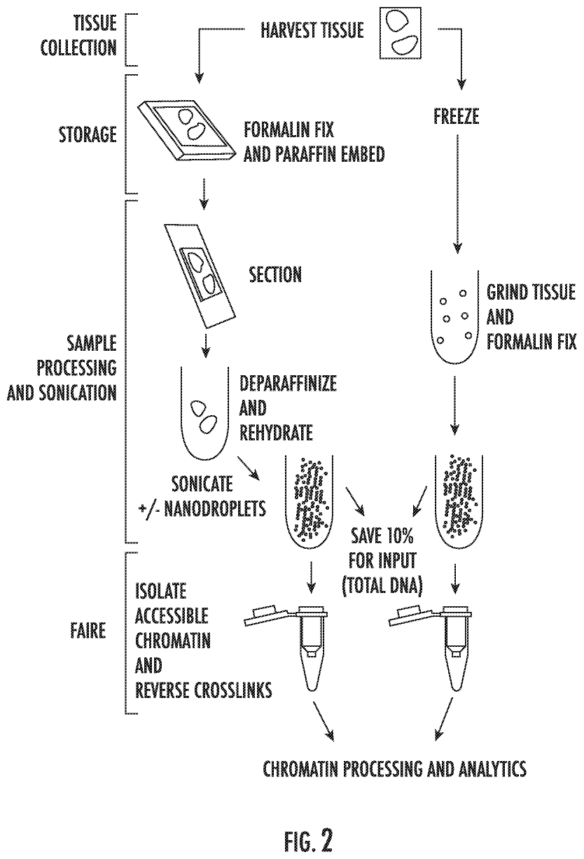

According to one aspect of the presently disclosed subject matter, methods are provided that involve using a cavitation enhancement agent, e.g., microbubbles, nanobubbles, and/or nanodroplets (e.g., phase-change nanodroplets), to decrease and/or eliminate oversonication-related issues and/or otherwise provide higher quality DNA fragments during the extraction of chromatin from tissue, e.g., as part of a FAIRE-type or other chromatin accessibility assay. FIG. 2 shows an exemplary assay from conducting FAIRE using nanodroplets during sonication. As shown at the top of FIG. 2, biological tissue, e.g., from a tumor or other disease tissue, can be harvested from a subject (e.g., a human or other mammalian subject) and stored, e.g., via formalin fixation and paraffin embedding or via freezing. When the FFPE tissue sample is ready to be analyzed, it can be sectioned, e.g., via microtome, deparaffinized (e.g., using an organic solvent, such as xylene), and rehydrated. Frozen samples can be ground and fixed with formalin (i.e., an aqueous formaldehyde solution). The sample can be sonicated in the presence of nanodroplets comprising, for example, a liquid perfluorocarbon core and an encapsulating shell, where the liquid core is converted to a gas via exposure to acoustic (e.g., ultrasound), energy, thus converting the nanodroplet into a microbubble or nanobubble that can aid in tissue dispersion. Following sonication, the sample is digested with RNase enzyme. About 10% of the sample is digested with proteinase K enzyme and the crosslinks are reversed in order to provide a total DNA input signal. Accessible fragments are biochemically isolated from the remaining sample by centrifugation through a silica matrix column or by phenol:chloroform extraction. Then, the isolated fragments can be assayed, e.g., via qPCR or HTS.

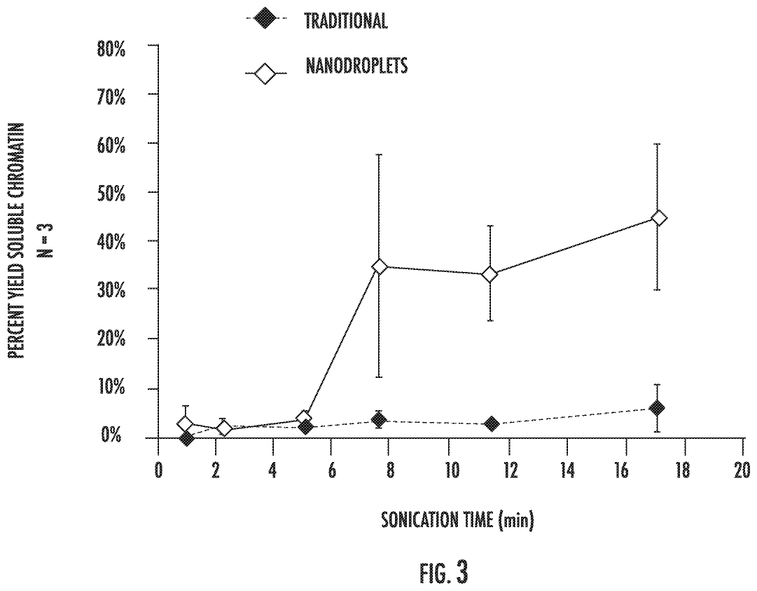

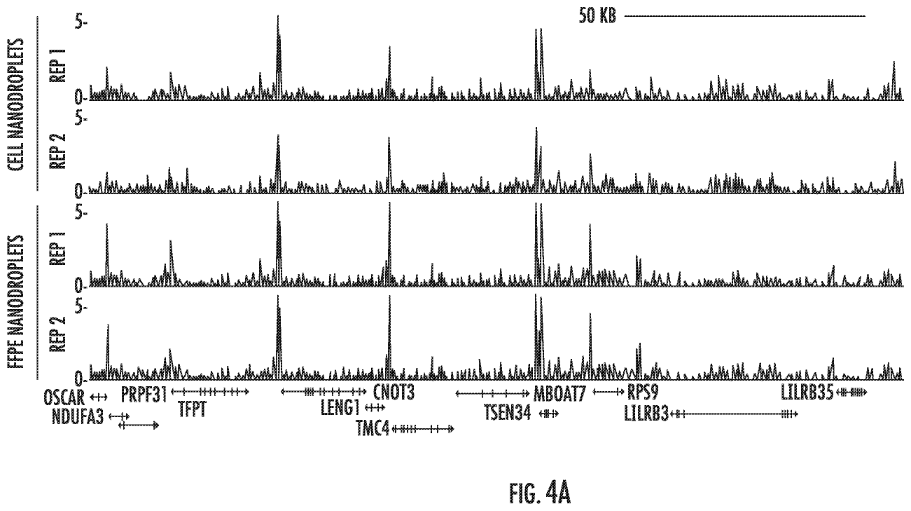

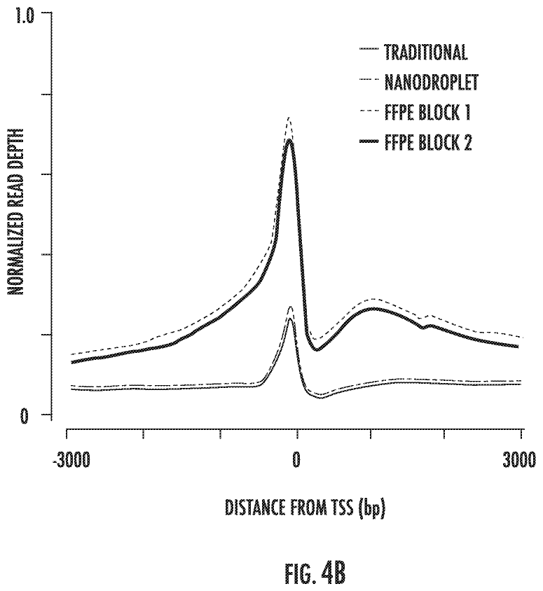

The increased soluble chromatin level (i.e., the increased level of DNA fragments from open chromatin regions) provided by the use of nanodroplet-assisted sonication is shown in FIG. 3. Further, the use of nanodroplet-assisted sonication does not alter the FAIRE-seq signal in xenograft tissue. See FIGS. 4A and 4B. As can be seen in FIG. 5, the use of nucleases in the enzymatic digestion of tissue samples results in a higher FAIRE background signal, which is indicative of low quality chromatin. The left-hand graph of FIG. 5, which provides data from samples digested with MNase, shows little difference between DNA signal from an inaccessible chromatin region and DNA signal from accessible chromatin regions, indicating a high background signal and digestion or loss of open chromatin from the assay. In contrast, the use of nanodroplet-assisted sonication provides much better signal-to-noise. See FIG. 5, right-hand graph. Signal from DNA associated with inaccessible chromatin regions is low and, thus easily distinguishable, compared to that from DNA associated with accessible regions.

Accordingly, in some embodiments, the presently disclosed subject matter provides a method of extracting chromatin from a biological tissue wherein the method includes exposing the biological tissue sample to ultrasonic energy (e.g., “sonicating” the tissue sample in an acoustic sonicator). The ultrasonic energy can comprise sound having any suitable frequency. In some embodiments, exposing the tissue sample to ultrasonic energy comprises exposing the tissue to sound having a frequency of between about 20 kilohertz (kHz) and about 2 megahertz (MHz). In some embodiments, exposing the sample to ultrasonic energy is performed using a “water bath” type sonicator and the sound has a frequency of between about 20 kHz and about 80 kHz (e.g., about 20, 30, 40, 50, 60, 70, or 80 kHz). In some embodiments, the sound has a frequency between about 20 kHz and about 40 kHz. In some embodiments, exposing the sample to ultrasonic energy is performed using a high-power focused sonicator and the sound has a frequency of between about 500 kHz and about 2 MHz (e.g., about 500, 600, 700, 800, 900, 1000, 1250, 1500, 1750, or about 2000 kHz). In some embodiments, the sound has a frequency of between about 1 MHz and about 2 MHz. In some embodiments, exposing the sample to ultrasonic energy is performed in conjunction with cooling the sample, e.g., by placing the sample in a cooling bath at a temperature below room temperature, such as in a bath at a temperature between about −10° C. and about 10° C., or between about 0° C. and about 10° C. Placing the sample in a cooling bath can counteract increases in temperature caused by the application of the acoustic energy to the sample.

The length of time that the tissue is exposed to the ultrasonic energy can vary depending upon the type of tissue, the type of analysis to be performed on the resulting DNA fragments obtained by exposing the tissue to ultrasonic energy, the optional use and type of other method steps performed on the tissue sample prior to the exposure to ultrasonic energy, the sound frequency used, and the optional presence of and/or type of and/or concentration of cavitation enhancement agents used during the exposure of the sample to ultrasonic energy. In some embodiments, the sample is sonicated for between about 15 seconds and about 40 minutes. In some embodiments, the sample is sonicated for between about 4 and about 10 minutes (e.g., about 4, 5, 6, 7, 8, 9, or about 10 minutes). In some embodiments, the sample can be sonicated for a period of time sufficient to shear the chromatin DNA to a suitable size for a particular application. For instance, the tissue sample can be sonicated for a period of time sufficient to provide soluble DNA fragments of between about 100 base pairs (bp) and about 900 bp. In some embodiments, e.g., when the DNA fragments are for use in a FAIRE assay (and analyzed via qPCR or HTS), the tissue sample can be sonicated for a period of time sufficient to provide soluble DNA fragments having an average size of between about 400 bp to about 900 bp or between about 400 bp and about 800 bp. In some embodiments, the sonication can be performed for a period of time sufficient to provide soluble DNA fragments having an average size of between about 600 bp to about 850 bp, or an average size of about 750 bp. In some embodiments, e.g., when the DNA fragments are for use in a ChIP-type assay, the average DNA fragments size can be smaller, and the sonication can be performed for a period of time sufficient to provide soluble DNA fragments having an average size of between about 100 bp and about 500 bp, or between about 200 bp and about 400 bp.

In some embodiments, a cavitation enhancement reagent is added to the tissue sample prior to or during exposure of the sample to the ultrasound energy. For example, a solution comprising the cavitation enhancement reagent can be added to the sample prior to or during sonication. Suitable cavitation enhancement agents include microbubbles (e.g., encapsulated microbubbles), nanobubbles, and phase-change nanodroplets. Suitable microbubbles, nanobubbles, and nanodroplets for use as part of the presently disclosed subject matter, and methods their production are described, for example, in U.S. Pat. No. 9,427,410 and U.S. Patent Application Publication No. 2015/0252355, each of which is incorporated herein by reference in its entirety.

More particularly, suitable microbubbles for use in the presently disclosed methods can include gas bubbles that are, in some embodiments, between about 1-10 microns in diameter. In some embodiments, suitable nanobubbles include gas bubbles that are between about 50 nm and about 999 nm in diameter, between about 100 nm and about 999 nm in diameter, or between about 500 nm and about 999 nm in diameter. In some embodiments, the microbubbles and/or nanobubbles comprise a compressible gas core encapsulated in a stabilizing shell, e.g. comprising one or more lipid, protein, peptide, gel, polymer, surfactant, sugar, another suitable encapsulating material, or a combination of such materials. In some embodiments, the shell comprises a lipid. Suitable lipids, include, but are not limited to, 1,2-distearoyl-sn-glycero-3-phosphocholine (DSPC), palmitoyl-2-hydroxy-sn-glycero-3-phosphocholine (LPC), and dipalmitoylphosphatidylcholine (DPPC), as well as conjugates of the lipids with synthetic polymers, such as a synthetic hydrophilic polymer, such as poly(ethylene glycol) (PEG). Suitable gases for use in the gas core include compounds that have a boiling point that is below room temperature (e.g., below about 20-25° C.) at one standard atmosphere of pressure. In some embodiments, the gas is a perfluorocarbon with a boiling point below room temperature, such as perfluorobutane (i.e., decafluorobutane (DFB)) or perfluoropropane (i.e., octafluoropropane (OFP)) or a combination thereof.

The compressible gas core of the microbubbles and nanobubbles enables them to compress and expand in a pressure field. For example, in an acoustic field, the bubble can compress and expand at the frequency of the sound wave. At moderate acoustic pressures, such as at about 2.4 MHz, the expansion and compression velocity of the microbubbles can be on the order of 350 meters per second. When the bubble remains intact, the expansion and compression of the bubble can be referred to as stable cavitation. At higher acoustic energies, the bubbles can oscillate to such a violent extent that the bubbles can break up, resulting in transient cavitation. At sufficient acoustic parameters, the microbubble-mediated cavitation can result in various effects, including disruption of cell membranes and shearing of DNA. See U.S. Pat. No. 9,982,290, incorporated herein by reference in its entirety.

In some embodiments, the cavitation enhancement agent comprises a nanodroplet. The nanodroplet can comprise a lipid or other encapsulating agent layer surrounding a liquid core. In some embodiments, the liquid core can convert to a gas upon exposure to the ultrasound energy. Once the liquid core converts to gas, the nanodroplet forms a microbubble or a nanobubble. In some embodiments, it forms a microbubble which has a radius several times larger than the initial nanodroplet. Suitable liquid core components include, but are not limited to hydrocarbons (e.g., isopentane), fluorocarbons, chlorofluorocarbons, hydroflurocarbons and perfluorocarbons, such as, perfluorobutane (i.e., decafluorobutane (DFB)), perfluoropropane (i.e., octafluoropropane (OFP)) perfluoropentane (i.e., dodeafluropentane (DDFP)), perfluorohexane (PFH), and perfluoroheptane.

In some embodiments, the liquid core comprises at least one perfluorocarbon (e.g., OFP or DFB) that has a boiling point below room temperature at one standard atmosphere of pressure. Although these materials would typically be expected to be gases at room temperature and standard pressure, methods of preparing these materials in stabilized droplet form have been previously described. See U.S. Pat. No. 9,427,410, incorporated herein by reference in its entirety. These “metastable” nanodroplets can be converted into gas microbubbles at temperatures close to zero with small amounts of ultrasound energy, thereby enabling effective nanodroplet-enhanced sonication of biological samples at temperatures that are low enough (e.g., a few degrees above freezing) to prevent temperature-related sample damage. In some embodiments, the nanodroplets comprise a liquid core comprising at least one perfluorocarbon that has a boiling point that is below room temperature at standard pressure (i.e., when the perfluorocarbon is not present in the nanodroplet core) and the liquid core of at least one or more of the nanodroplets remains liquid for at least one hour at room temperature at standard pressure.

As an alternative to adding a solution of microbubbles, nanobubbles, and/or nanodroplets, in some embodiments, a cavitation enhancement agent (e.g., microbubbles and/or nanobubbles) can be generated in the sample when the sample is exposed to the ultrasound energy. For example, in some embodiments, a microbubble-generation and/or nanobubble-generation substrate, such as a material comprising a rough surface, can be placed in the sample and can provide a surface upon which microbubbles and/or nanobubbles are nucleated upon sonication of the sample. The rough surface provides microscopic holes or cavities which are favorable locations for dissolved gas molecules to form microbubbles and/or nanobubbles, which can then escape into the fluid. Theory related to the formation of the microbubbles on surfaces is described, for example, in Atchley and Prosperetti, The Journal of the Acoustical Society of America, 86(3), 1065-1084 (1989); and Crum, Nature, 278, 148-149 (1979). The rough surface can be on a surface added to the sample before or when it is exposed to ultrasonic energy or the rough surface can be on the interior of the container which holds the sample itself. For instance, such rough materials can include plastic rods or particles.

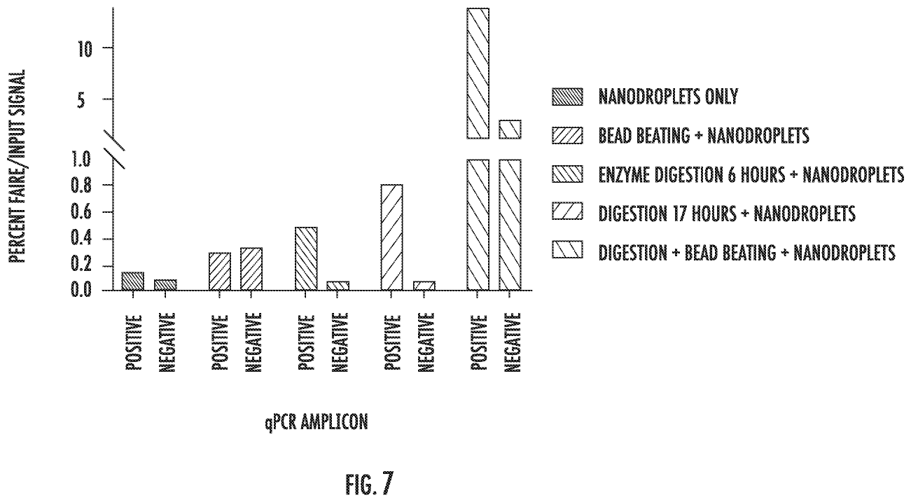

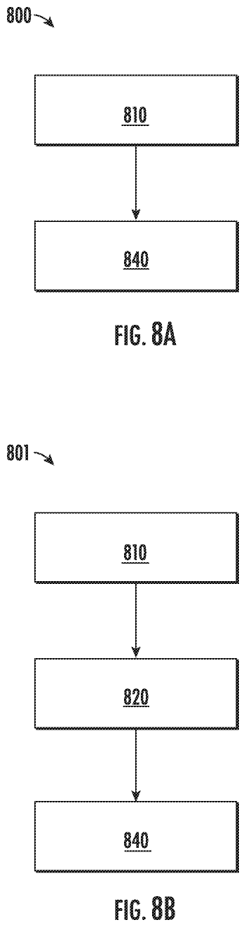

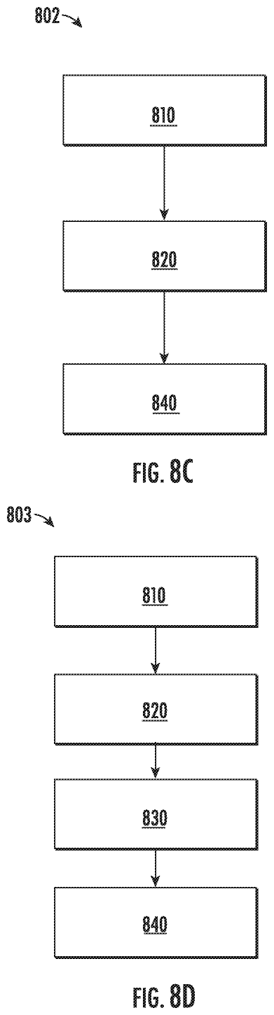

FIG. 8A is a flow chart illustrating method 800 for extracting chromatin from a tissue sample according to one embodiment of the presently disclosed subject matter. In the embodiment illustrated in FIG. 8A, a biological tissue sample is received (step 810). Receiving step 810 can also include initial processing of the sample. The initial processing can vary depending upon the type of biological sample received. For example, in some embodiments, the tissue sample is a FFPE tissue sample and step 810 includes removing paraffin from the tissue using a suitable organic solvent, e.g., xylene or another aromatic organic solvent (e.g., toluene, benzene, etc.), an aliphatic hydrocarbon, such as hexanes or pentanes, a non-polar organic ether, such as tetrahydrofuran (THF) or diethyl ether, or any mixture thereof. Step 810 can further include rehydrating the tissue, e.g., by exposing the deparaffinized tissue sample first to a solution comprising 100% alcohol (e.g., ethanol) and then to a series of decreasingly concentrated alcohol baths. Alternatively, if the tissue sample is a frozen tissue sample, the sample can be thawed. Also in step 810, e.g., prior to any paraffin removal, the tissue sample can be sectioned to provide samples of a desired size. For instance, thinner samples of the original tissue sample can be prepared by slicing the original sample using a microtome. Following step 810, the sample is exposed to ultrasonic energy as described hereinabove in sonication step 840, optionally in the presence of a cavitation enhancement reagent or in the presence of a microbubble-generation substrate. For example, in some embodiments, step 840 includes adding a solution of microbubbles, nanobubbles, and/or nanodroplets to the sample and exposing the sample to ultrasonic energy. As described above, in some embodiments, the sample is placed in a cooling bath during step 840. Thus, in some embodiments, the presently disclosed subject matter provides a method comprising (a) receiving a sample comprising a biological sample, wherein the receiving can optionally include removing paraffin from the sample or thawing the sample and/or slicing the sample; and (b) exposing the sample to ultrasound energy, thereby providing a processed sample comprising chromatin fragments that have been extracted from the biological tissue.

The tissue sample can be any suitable biological tissue sample. In some embodiments, the tissue sample is from a mammal. In some embodiments, the tissue sample is from a human. In some embodiments, the tissue sample is a FFPE sample. In some embodiments, the tissue sample is from an individual who has been diagnosed with a disease (e.g., cancer) or who is suspected of having a disease. In some embodiments, the sample is a tumor sample or a suspected tumor sample.

In some embodiments, the presently disclosed methods can further include one or more additional steps that can be performed after the initial receiving and paraffin removal or thawing of the sample (step 810 of FIG. 8A) and the sonication step (step 840 of FIG. 8A). For example, in some embodiments, e.g., when the sample includes a human tissue or another type of tissue that includes a relatively higher level of extracellular material, the presently disclosed subject matter can further include a mechanical dissociation step to help to break down the tissue prior to the sonication step. Suitable mechanical dissociation for use according to the presently disclosed subject matter include, for instance, a bead beating step or the use of a motorized or non-motorized dounce tissue homogenizer or probe sonicator. Bead beating can include the use of any suitable size or type of beads, including, but not limited to, ceramic beads, glass beads, zirconia beads, silica beads, chrome-steel beads, stainless steel beads, silicon carbide beads, garnet beads, or tungsten carbide beads. The mechanical dissociation step can be performed for a period of time sufficient to start breaking up the extracellular matrix, e.g., to cause the sample to dissociate. For instance, mechanical dissociation can result in one or more large pieces of tissue being dispersed into the component cells or small pieces of tissue in solution. The time to dissociation can range from about 10 seconds to about 10 minutes (e.g., about 10, 15, 30, 45, or 60 seconds or about 2, 3, 4, 5, 6, 7, 8, 9, or 10 minutes).

FIG. 8B is a flow chart illustrating method 801 for extracting chromatin from a tissue sample according to one embodiment of the presently disclosed subject matter. Steps 810 and 840 are the same as those described for method 800 of FIG. 8A. Step 820 is a step wherein, for example, following paraffin removal and rehydration of a FFPE tissue sample in step 810, the rehydrated tissue (e.g., the rehydrated sliced tissue) is exposed to bead beating or another mechanical dissociation technique for a period of time prior to sonication step 840. Thus, in some embodiments, the presently disclosed subject matter provides a method comprising (a) receiving a sample comprising a biological sample, wherein the receiving can include (a1) optionally removing paraffin from the sample or thawing the sample and/or slicing the sample, and (a2) performing a mechanical dissociation step, such as bead beating or using a tissue homogenizer or probe sonicator; and (b) exposing the sample to ultrasound energy, thereby providing a processed sample comprising chromatin fragments that have been extracted from the biological tissue.

In some embodiments, e.g., when the tissue sample is a human tissue sample or other tissue sample comprising a relatively higher amount of extracellular material, the presently disclosed subject matter can further include an enzyme digestion step to help to break down the tissue prior to the sonication step. In particular, the enzyme digestion step can be used to break down extracellular matrix components in the tissue prior to sonication and shearing of the chromatin. Thus, in some embodiments, the sample is contacted with an enzymatic solution comprising one or more enzymes that digest one or more extracellular matrix component, such as collagen or hyaluronic acid (HA). For example, in some embodiments, the enzymatic solution comprises a collagenase (e.g., a matrix metallopeptidase (MMP)) and/or a hyaluronidase. In some embodiments, the collagenase or hyaluronidase is a mammalian collagenase or hyaluronidase (e.g., a rat, mouse, rabbit or human collagenase or hyaluronidase). The collagenase can be a fibroblast/interstitial collagenase (i.e., collagenase 1 or MMP-1), a neutrophil collagenase (i.e., collagenase 2 or MMP-8) or collagenase type 3 (i.e., MMP-13). In some embodiments, the enzymatic solution comprises a mixture of collagenases and/or a mixture of hyaluronidases. In some embodiments, the enzymatic solution can include one or more additional proteolytic enzymes. In some embodiments, the enzymatic solution is free of proteinase K.

As described above with regard to FIG. 5, the use of MNase can result in enzymatic bias and/or a lower signal-to-noise in a chromatin signature assay. Accordingly, in some embodiments, the enzymatic solution of the pre-sonication enzymatic digestion step is free of MNase, DNase, Tn5 transposase, Nt.CViPII, and/or M.CvPI. In some embodiments, the enzymatic solution is free of any endonuclease. In some embodiments, the enzymatic solution is free of any DNA methyltransferase. In some embodiments, the enzymatic solution is free of any nuclease.

The contacting of the sample and the enzymatic solution can be performed at any suitable temperature with or without mixing during incubation. In some embodiments, the temperature is selected based on the optimal temperature for the enzyme or enzymes being used. Collagenases generally have optimal activity in a pH range of about 6.3 to about 8.5, while hyaluronidase generally has optimal activity in a somewhat lower pH range, e.g., between about 4.5 and 7. In some embodiments, the contacting can take place in a suitable buffer (e.g., having a pH between about 6 and about 8.5) at a temperature above about 20° C. In some embodiments, the temperature is between about 32° C. and about 40° C. In some embodiments, the temperature is about 37° C.

The contacting can be performed for a period of time sufficient to start breaking up the extracellular matrix, e.g., to cause the sample to be less viscous. For example, in some contacting is performed for between about 4 hours and about 17 hours (e.g., about 4, 5, 6, 7, 8, 9, 10, 11, 12, 13, 14, 15, 16, or about 17 hours). At the end of that time, a suitable chelator (e.g., EDTA) or pH adjusting agent can be added to the sample to quench the enzymatic reaction or reactions.

FIG. 8C is a flow chart illustrating method 802 for extracting chromatin from a tissue sample according to an embodiment of the presently disclosed subject matter. Steps 810 and 840 are the same as those described for method 800 of FIG. 8A. Step 830 is a step wherein, for example, following paraffin removal and rehydration of a FFPE tissue sample in step 810, the rehydrated tissue (e.g., the rehydrated sectioned tissue) is exposed to an enzymatic solution comprising one or more enzymes that digest one or more extracellular matrix components, such as a solution comprising one or more collagenase and one or more hyaluronidase. Thus, in some embodiments, the presently disclosed subject matter provides a method comprising (a) receiving a sample comprising a biological sample, wherein the receiving can optionally include removing paraffin from the sample or thawing the sample; (b) contacting the sample with an enzymatic solution comprising an extracellular matrix digestion enzyme; and (c) exposing the sample to ultrasound energy, thereby providing a processed sample comprising chromatin fragments that have been extracted from the biological tissue.

FIG. 8D is a flow chart illustrating method 803 for extracting chromatin from a tissue sample according to an embodiment of the presently disclosed subject matter. Steps 810 and 840 are the same as those described for method 800 of FIG. 8A. Step 820 is the same as that described for method 801 of FIG. 8B, and step 830 is the same as that described for method 802 of FIG. 8C. Thus, in some embodiments, the presently disclosed subject matter provides a method comprising (a) receiving a sample comprising a biological sample, wherein the receiving can include (a1) optionally removing paraffin from the sample or thawing the sample and/or sectioning the sample and (a2) performing a mechanical dissociation step; (b) contacting the sample with an enzymatic solution comprising an extracellular matrix digestion enzyme; and (c) exposing the sample to ultrasound energy, thereby providing a processed sample comprising chromatin fragments that have been extracted from the biological tissue.

The processed sample can be analyzed or assayed in any suitable manner. For example, in some embodiments, the processed sample can be assayed using qPCR or HTS. Thus, in some embodiments, the processed method can be part of a modified FAIRE-type assay or part of a ChIP-type assay. In some embodiments, the presently disclosed methods are free of the use of an antibody.

In some embodiments, the presently disclosed methods provide a processed sample wherein the chromatin fragments derived from accessible chromatin in the tissue sample are quantifiably distinguishable from chromatin fragments derived from inaccessible chromatin in the tissue sample. In some embodiments, the presently disclosed methods provide a processed sample wherein chromatin fragments derived from precipitation of a protein crosslinked to chromatin are distinguishable from chromatin fragments that do not contain the protein. Precipitation of the protein can be performed using an immunoprecipitation technique (e.g., using an antibody that binds the protein) or a chemical precipitation technique. Thus, the presently disclosed methods can preserve the chromatin signature. In some embodiments, the difference between a detectable signal generated by the chromatin fragments derived from accessible chromatin or generated from chromatin fragments derived from precipitation of a protein crosslinked to chromatin are at least about 1.1, 1.2, 1.3, 1.4, or about 1.5 times that of the signal generated by other chromatin in the sample.

In some embodiments, the amount of and/or the detectable signal generated from the chromatin fragments derived from the accessible chromatin is at least about 1.5 times or more than the amount of and/or detectable signal generated from the inaccessible chromatin. For example, in some embodiments, the method provides a detectable signal (e.g., a qPGCR signal) from an accessible chromatin region that is about 2, 2.5, 3, 3.5, 4, 4.5, 5, 5.5, 6, 6.5, 7, 7.5, 8, 8.5, 9, or 10 times more the detectable signal from an inaccessible region.

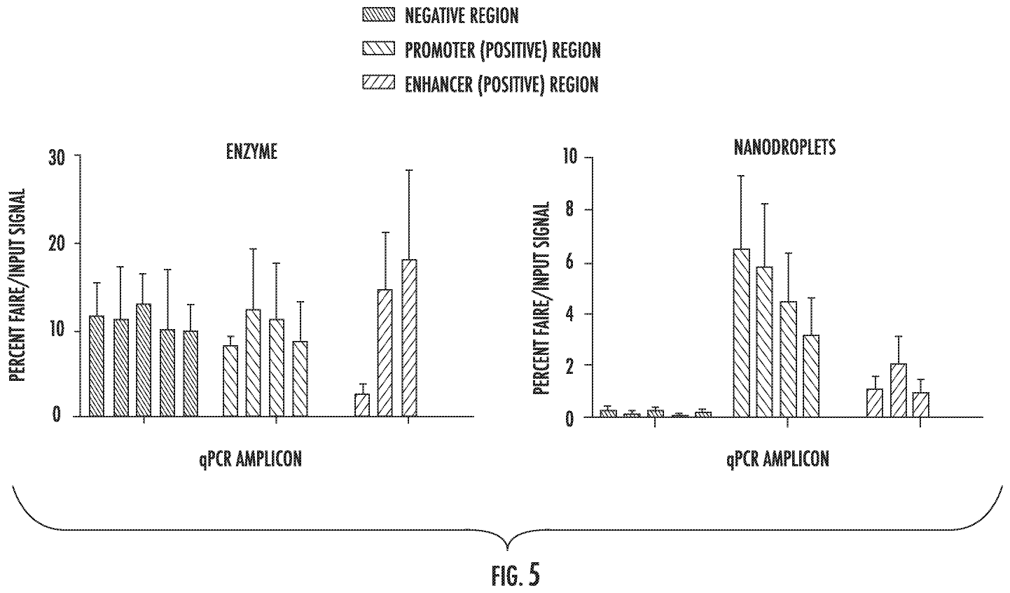

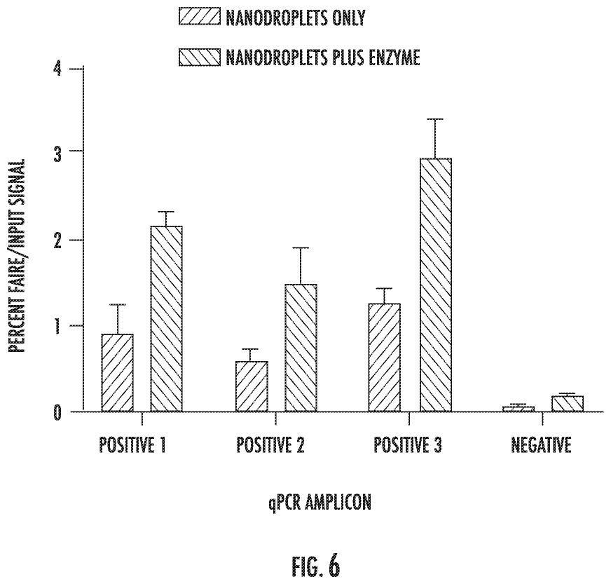

FIGS. 6 and 7 illustrate the effects of adding further method steps to those described for method 800 in FIG. 8A on signal-to-noise of a qPGCR signal. FIG. 6 shows the percent FAIRE/input signal associated with open chromatin regions (i.e., Positive 1, Positive 2, Positive 3) and an inaccessible chromatin region (i.e., Negative) when enzyme digestion using collagenase and hyaluronidase is performed prior to nanodroplet-assisted sonication and when enzyme digestion is not used prior to nanodroplet-assisted sonication. FIG. 7 shows the percent FAIRE/input signal associated with an open chromatin region (i.e., Positive) and an inaccessible chromatin region (i.e., Negative). More particularly, FIG. 7 shows the effects of adding a bead beating mechanical dissociation step (“Bead Beating+Nanodroplets”) to that of the method 800 (“Nanodroplets only”). FIG. 7 also shows the effects of adding an enzymatic digestion step using collagenase and hyaluronidase for 6 or 17 hours (“Digestion 6 Hours+Nanodroplets” and “Digestion 17 Hours+Nanodroplets”) as well as that of adding both an enzymatic digestion step and a bead beating step (“Digestion+Bead Beating+Nanodroplets”). As shown in FIGS. 6 and 7, the use of additional steps, particularly an enzymatic digestion step that uses enzymes that target the extracellular matrix, can provide improved signal-to-noise.

### III. Kits for Chromatin Extraction/Assays

In some embodiments, the presently disclosed subject matter provides a kit for use in extracting and/or assaying chromatin (e.g., accessible chromatin). Thus, in some embodiments, the kit can be for used in conjunction with one of the presently disclosed methods.

In some embodiments, the presently disclosed subject matter provides a kit comprising: (a) an extracellular matrix (ECM) digestion solution comprising one or more enzymes that digest an extracellular matrix component; and (b) a cavitation enhancement agent. In some embodiments, the ECM digestion solution comprises a collagenase and/or a hyaluronidase.

The kit can also include one or more additional components. In some embodiments, the kit can include one or more types or sizes of beads for use in a bead beating step. Thus, in some embodiments, the kit can comprise beads, including but not limited to, ceramic beads, glass beads, zirconia beads, silica beads, chrome-steel beads, stainless steel beads, silicon carbide beads, garnet beads, and/or tungsten carbide beads of any diameter.

In some embodiments, the kit can include an organic solvent or organic solvent mixture for use in dissolving paraffin. The organic solvent or solvent mixture can include any suitable nonpolar organic solvent or solvents, such as, but not limited to, an ether (e.g., THF), an aliphatic hydrocarbon, an aromatic solvent (e.g., xylenes), or a mixture thereof. In some embodiments, the kit can further include one or more rehydration solutions (e.g., solutions that contain different volume percentages of an alcohol (e.g., ethanol) in water or an aqueous solution).

In some embodiments, the ECM digestion solution is free of one or more of micrococcal nuclease (MNase), DNase, Tn5 transposase, Nt.CviPII and M.CviPI. In some embodiments, the ECM digestion solution is free of an endonuclease and/or a DNA methyltransferase. In some embodiments, the ECM digestion solution is free of a nuclease. In some embodiments, the ECM digestion solution is free of proteinase K. The kit can, however, include one or more of these types of enzymes (e.g., an RNase or proteinase K) as part of one or more solutions that can be added to a sample after sonication.

In some embodiments, the cavitation enhancement reagent comprises nanodroplets. In some embodiments, the nanodroplets comprise a liquid core comprising a perfluorocarbon, a fluorocarbon, a chlorofluorocarbon, a hydrofluorocarbon, a hydrocarbon or a mixture thereof. In some embodiments, the nanodroplets comprise a lipid or other encapsulating layer surrounding the liquid core (e.g., prior to exposure of the nanodroplet to ultrasonic energy). In some embodiments, the nanodroplets comprise a perfluorocarbon, optionally wherein the perfluorocarbon comprises has a boiling point below about room temperature at one standard atmosphere of pressure and/or wherein the perfluorocarbon is decafluorobutane and/or octafluoropropane. In some embodiments, at least a portion of the nanodroplets are able to vaporize to form microbubbles and/or nanobubbles upon exposure to the ultrasonic energy. In some embodiments, the nanodroplets comprise a liquid core comprising a perfluorocarbon that has a boiling point that is below room temperature at one standard atmosphere of pressure when the perfluorocarbon is not present in the liquid core of the nanodroplet and wherein the liquid core of at least one or more of the nanodroplets remains liquid for at least one hour at room temperature at one standard atmosphere of pressure.

In some embodiments, the cavitation enhancement agent comprises microbubbles and/or nanobubbles. In some embodiments, the microbubbles and/or nanobubbles comprise a gaseous core encapsulated in a stabilizing shell (e.g., comprising a lipid, a surfactant, a polymer, a peptide, a protein or another suitable encapsulating material). In some embodiments, the gaseous core comprises a perfluorocarbon gas. In some embodiments, the perfluorocarbon gas comprises decafluorobutane and/or octafluoropropane.

In some embodiments, the cavitation enhancement agent comprises a microbubble-generation and/or nanobubble-generation substrate, such as a rod or particle comprising microscopic holes and/or cavities. These holes and/or cavities are favorable locations for dissolved gas molecules to form microbubbles and/or nanobubbles, which can then escape into the surrounding fluid. These hole and/or cavities can also be on the interior surface of the vessel used to contain the tissue sample when it is exposed to ultrasonic energy. Theory related to the formation of the microbubbles is described, for example, in Atchley and Prosperetti, The Journal of the Acoustical Society of America, 86(3), 1065-1084 (1989); and Crum, Nature, 278, 148-149 (1979).

In some embodiments, the kit can further include one or more buffers for use during a method of the presently disclosed subject matter, a chelator for quenching the ECM digestion solution, and/or one or more PCR primers. In some embodiments, the kit can include one or more of a commercially available PCR master mix (e.g., including enzyme, nucleotides, a dye, such as that sold under the tradename SYBR™ (Molecular Probes, Inc., Eugene, Oreg., United States of America), magnesium, and buffer), collection tubes, protease inhibitor cocktail, silica matrix DNA clean and concentrate columns, sonication buffer, and/or a FAIRE or ChIP assay buffer.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter.

### Example 1

### FFPE Tissue Digestion Protocol

Store FFPE tissue mounted slides at −80° C. inside of a plastic cassette wrapped in tinfoil and sealed airtight within a plastic bag. Control for moisture by placing a pouch of desiccants within the cassette. Remove the FFPE slides from the −80° C. freezer and allow to cool to room temperature prior to opening the plastic bag.
1. To maximize chromatin recovery, utilize at least 4 sections each 10 m in size. Remove paraffin by placing the FFPE tissue on a slide holder and transferring to a coupling jar containing 200 ml of fresh xylene. Incubate for 3 minutes and blot dry on a paper towel. Repeat for a total of 3 xylene treatments.
2. While incubating in xylene, prepare 2 coupling jars containing 200 ml of 100% ethanol, 2 jars containing 85% ethanol, 1 jar containing 70% ethanol, 1 one jar containing double deionized water. After the third xylene treatment, incubate the slides sequentially for 3 minutes in the decreasingly concentrated ethanol water baths as previously listed.
3. Remove the slides from the double deionized water and blot the tissue dry with a paper towel. Carefully use a fine razor blade to scrape the tissue horizontally off the slide in one fluid motion. The tissue should remain intact and rest on top of the slide. Hold the slide up to the top of a 1.5 mL microtube and use the razor to brush the tissue carefully into the tube.
4. Prepare a fresh enzymatic digestion cocktail containing 10 units/ml type 3 collagenase (Worthington Biochemical Corporation, Lakewood, N.J., United States of America; Cat No. LS004182, Lot No. 44B14775), 80 units/ml pure collagenase (Worthington Biochemical Corporation, Lakewood, N.J., United States of America; Cat No. LS005273, Lot No. 56E16614), and 100 units/ml hyaluronidase (Worthington Biochemical Corporation, Lakewood, N.J., United States of America, Cat No. LS005475, Lot No. 57A17191). Aliquots of the enzymes can each be stored at −20° C. protected from light in Hanks Balanced Salt Solution (HBSS) (Gibco Laboratories, Gaithersburg, Md., United States of America; Ref No. 14175-095) supplemented with 0.14 g/L calcium chloride, 0.10 g/L magnesium chloride, and 0.10 g/L magnesium sulfate. Prepare 1 ml of HBSS and enzyme cocktail for each tissue and suspend the tissue pellet. Digest for at 37° C. on a rotating platform for 8 hours.
5. Remove from the rotating platform and quench the reaction by adding EDTA, pH 8.0 to a final concentration of 1 mM into each tube and briefly vortexing.
6. Centrifuge at 1,200×g for 5 minutes, then discard the supernatant.
7. Add 90 μl of Formaldehyde Assisted Isolation of Regulatory Elements (FAIRE) buffer (10 mM Tris-HCL, pH 8.0, 2% Triton-X-100, 1% SDS, 100 mM NaCl, and 1 mM EDTA; see Giresi et al., Genome Research, 17, 877-885 (2007)) per tissue and pipette lightly to resuspend the pellet taking care not to create foam.
8. Transfer solution to borosilicate glass tubes (Thermo Fisher Scientific, Waltham, Mass., United States of America; C4008-632R). Add 10 μl of nanodroplets for a total volume of 100 μl. Seal the tube with a crimp cap (Thermo Fisher Scientific, Waltham, Mass., United States of America; C4008-6A), invert the tube horizontally in a circular motion to mix, and briefly spin down.
9. Sonicate the solution-containing vials in the Covaris E110 Sonicator (Covaris, Woburn, Mass., United States of America) utilizing a custom holder at 4° C. with the following settings: 20% duty cycle; intensity=8; and 200 cycles per burst, for 8 minutes. Sonication time may need to be optimized for FAIRE and may vary depending upon tissue type. (Note: When using Covaris E110 Sonicator, sonicate samples for 4 minutes maximum at a time to avoid overheating)
10. Remove from the sonicator and centrifuge full speed (>20,000×g) at 4° C. for 5 minutes. Discard pellets and transfer supernatants into PCR tubes.
11. Add 2 μl [100 mg/mL] RNase (Qiagen, Cat No. 19101) to all samples, invert to mix, briefly spin down, and incubate at 37° C. for 10 minutes inside a thermocycler (e.g., a thermocycler sold under the tradename BioRad T100™ ThermalCycle (Bio-Rad, Hercules, Calif., United States of America)).
12. Combine the supernatants from all sections into one 1.5 ml microtube and pipette to mix.
13. Transfer 10% (approximately 40 μl if starting with 4 sections) of the supernatant from each tube to a new PCR tube to serve as inputs. Bring the total volume of input up to 100 μl through the addition of FAIRE buffer.
14. Add 2 μl proteinase K to the input tubes. Invert to mix and briefly centrifuge. Incubate inside the thermocycler at 55° C. for 1 hour followed by 80° C. for 2 hours. Leave at 4° C. overnight
15. Perform FAIRE on the remaining 90% supernatant using the Zymo Research ChIP DNA Clean and Concentrator kit (Zymo Research, Irvine, Calif., United States of America; Cat No. 11-379) according to manufacturer's instructions and elute in a final volume of 25 μL. Place eluted DNA in fridge when done.
16. On the next day, extract the DNA from the inputs using the Zymo Research ChIP DNA Clean and Concentrator kit (Zymo Research, Irvine, Calif., United States of America; Cat No. 11-379) according to manufacturer's instructions and elute in a final volume of 25 μl.

### Other Permutations

Use of microbubbles and/or nanobubbles with polymer or protein shells during sonication
Use of microbubbles and/or nanobubbles with different gas cores, such as oxygen, air or different perfluorocarbons
Use of phase-change nanodroplets which convert to microbubbles and/or nanobubbles upon application of sonic energy
Use of microbubbles and/or nanobubbles with different sizes
Use of varying acoustic energies, duty cycles, and frequencies to optimize interaction with microbubbles and/or nanobubbles or DNA fragment size
Use of microfluidics systems to produce microbubbles and/or nanobubbles directly at the site of use

### Example 2

### Preparation of Lipid Coated Microbubbles

Lipid coated microbubbles can be prepared as described in U.S. Patent Application Publication No. 2015/0252355. Briefly, lipid monolayer-coated microbubbles were prepared using 1,2-distearoyl-snglycero-3-phosphocholine (DSPC)(Avanti Polar Lipids, Alabaster, Ala., United States of America) and 1,2-distearoyl-sn-glycero-3-phosphoethanolamine-N-methoxy (polyethylene-glycol)-2000 (DSPE-PEG2000) (Avanti Polar Lipids, Alabaster, Ala., United States of America) in a 9 to 1 molar ratio as previously described. The lipids were dissolved in a buffer solution comprised of phosphate-buffered saline (PBS), propylene glycol, and glycerol (16:3:1) for a total lipid concentration of 1.0 mg/mL. The resulting lipid solution was placed into 3 mL glass vials in 1.5 mL aliquots. The vials were sealed with rubber septa and 5 capped. Finally, the air in the vial headspace was removed via a custom vacuum apparatus and replaced with decafluorobutane (Fluoromed, Round Rock, Tex., United States of America). The vial was shaken vigorously for 45 seconds using a high-speed mixer (Vialmix, Bristol-Myers Squibb Medical Imaging, North Billerica, Mass., United States of America) to produce a polydisperse distribution (Mean Diameter: 1.07±0.9, Concentration: 1.1×10¹⁰ bubbles per ml).

The disclosure of each of the publications referenced herein is incorporated herein by reference in its entirety.

It will be understood that various details of the presently disclosed subject matter may be changed without departing from the scope of the presently disclosed subject matter. Furthermore, the foregoing description is for the purpose of illustration only, and not for the purpose of limitation.

1. A method of extracting chromatin from tissue, the method comprising:
(a) receiving a sample comprising a biological tissue;
(b) contacting the sample with an enzymatic solution, wherein the enzymatic solution comprises one or more enzymes that digest one or more extracellular matrix components; and
(c) exposing the sample to ultrasound energy, thereby providing a processed sample comprising chromatin fragments that have been extracted from the biological tissue.

2. The method of claim 1, wherein the sample is from a formaldehyde fixed paraffin embedded (FFPE) tissue, and wherein the receiving further comprises initially processing the sample, wherein the initial processing comprises: contacting the FFPE tissue with a solution comprising an organic solvent, wherein the organic solvent is a nonpolar organic solvent, and wherein the nonpolar organic solvent is selected from an ether, an aliphatic hydrocarbon, or an aromatic hydrocarbon.

3. (canceled)

4. (canceled)

5. (canceled)

6. (canceled)

7. (canceled)

8. (canceled)

9. The method of claim 1, wherein exposing the sample to ultrasound energy comprises exposing the sample to a sound having a frequency of between about 20 kilohertz (kHz) and about 2 megahertz (MHz).

10. (canceled)

11. The method of claim 1, wherein a solution comprising a cavitation enhancement agent is added to the sample prior to or during exposure of the sample to the ultrasound energy.

12. The method of claim 11, wherein the cavitation enhancement reagent comprises nanodroplets.

13. (canceled)

14. (canceled)

15. (canceled)

16. The method of claim 12, wherein the nanodroplets comprise a liquid core comprising a perfluorocarbon that has a boiling point that is below room temperature at one standard atmosphere of pressure when the perfluorocarbon is not present in the liquid core of the nanodroplet and wherein the liquid core of at least one or more of the nanodroplets remains liquid for at least one hour at room temperature at one standard atmosphere of pressure.

17. (canceled)

18. (canceled)

19. (canceled)

20. (canceled)

21. The method of claim 1, wherein the processed sample comprises a soluble DNA peak fragment size of between about 100 base pairs (bp) and about 900 bp.

22. The method of claim 21, wherein the soluble DNA peak fragment size is between about 100 bp and about 500 bp.

23. The method of claim 21, wherein the soluble DNA peak fragment size is between about 400 bp and about 800 bp.

24. The method of claim 1, wherein the method is free of the use of an antibody.

25. The method of claim 1, wherein the method provides a processed sample wherein chromatin fragments derived from accessible chromatin in the tissue sample are quantifiably distinguishable from chromatin fragments derived from inaccessible chromatin in the tissue sample or a processed sample wherein chromatin fragments derived from precipitation of a protein crosslinked to chromatin are distinguishable from chromatin fragments that do not contain the protein.

26. The method of claim 25, wherein the amount of and/or the detectable signal generated from chromatin fragments derived from the accessible chromatin is at least about 1.5 times or more than the amount of and/or detectable signal generated from chromatin fragments derived from the inaccessible chromatin, and wherein the chromatin fragments in the processed sample are assayed using quantitative PCR or high throughput sequencing.

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. A kit for extracting chromatin from a tissue, the kit comprising:
(a) an extracellular matrix (ECM) digestion solution comprising one or more enzymes that digest an extracellular matrix component, wherein the one or more enzymes comprise collagenase and/or hyaluronidase; and
(b) a cavitation enhancement agent.

53. The kit of claim 52, further comprising ceramic beads, glass beads, zirconia beads, silica beads, chrome-steel beads, stainless steel beads, silicon carbide beads, garnet beads, and/or tungsten carbide beads.

54. The kit of claim 52, further comprising an organic solvent that can dissolve paraffin, optionally wherein the organic solvent is a nonpolar organic solvent and wherein the organic solvent comprises an ether, an aliphatic hydrocarbon, or an aromatic solvent.

55. The kit of claim 52, wherein the ECM digestion solution is free of micrococcal nuclease, DNase, Tn5 transposase, Nt.CviPII and/or M.CviPI, wherein the ECM digestion solution is free of an endonuclease and/or a DNA methyltransferase and wherein the ECM digestion solution is free of a nuclease.

56. The kit of claim 52, wherein the ECM digestion solution is free of proteinase K.

57. The kit of claim 52, wherein the cavitation enhancement reagent comprises nanodroplets.

58. (canceled)

59. The kit of claim 57, wherein the nanodroplets comprise a lipid layer surrounding a liquid core prior to exposure to the ultrasonic energy.

60. (canceled)

61. The kit of claim 57, wherein the nanodroplets comprise a liquid core comprising a perfluorocarbon that has a boiling point that is below room temperature at one standard atmosphere of pressure when the perfluorocarbon is not present in the liquid core of the nanodroplet and wherein the liquid core of at least one or more of the nanodroplets remains liquid for at least one hour at room temperature at one standard atmosphere of pressure.

62. (canceled)

63. (canceled)
